(19) United States
(12) Patent Application Publication
(10) Pub. No.: US 2007/0128300 A1
(43) Pub. Date: Jun. 7, 2007

(54) Natural tea having effects of hangover relief and liver function recovery and method for preparing the same

(76) Inventor: Jong Hyun Nam, Seoul (KR)

Correspondence Address: ST. ONGE STEWARD JOHNSTON & REENS, LLC, 986 BEDFORD STREET, STAMFORD, CT, 06905-5619, US

(21) Appl. No.: 11/494,922

(22) Filed: Jul. 28, 2006

Related U.S. Application Data
(63) Continuation of application No. PCT/KR04/01050, filed on May 6, 2004.

(30) Foreign Application Priority Data
Jan. 29, 2004 (KR) 10-2004-0005848

(51) Int. Cl. A61K 36/48

(52) U.S. Cl. 424/757; 424/773

(57) ABSTRACT

A natural tea has effects of relieving hangovers and revitalizing liver functions, contains an extract from natural plant Robinia pseudo-acacia as a main active ingredient, and is obtained under optimal extraction conditions. The natural tea comprises an extract from the leaf, stem, flower and root of Robinia pseudo-acacia as a main active ingredient, and conventional additives, and optionally, at least one selected from Hedyotis diffusa, Amomi semen, Glycyrrhiza and Pueraiae Flos, as a minor active ingredient. Thus, the present invention provides the powder or liquid natural tea comprising the Robinia pseudo-acacia extract as a main active ingredient, and optionally, a given amount of at least one selected from the Hedyotis diffusa, Amomi semen, Glycyrrhiza and Pueraiae Flos extracts, as well as the method for preparing the same. The natural tea according to the present invention shows the effects of relieving hangovers and protecting the liver when taken before or after drinking alcohol. Particularly, according to the present invention, robinine extracted from Robinia pseudo-acacia can be used as a single active ingredient in a hangover relief drink.

### CROSS-REFERENCE TO RELATED APPLICATIONS

The present application is a continuation of pending International Patent Application PCT/KR2004/001050 filed on May 6, 2004 which designates the United States and claims priority of Korean Patent Application No. 10-2004-0005848 filed on Jan. 29, 2004.

### FIELD OF THE INVENTION

The present invention relates to a type of natural tea having the effects of relieving hangovers and revitalizing liver functions, and a method for preparing the same. More particularly, the present invention relates to a natural tea having the effects of relieving hangovers and revitalizing liver functions, which contains an extract from Robinia pseudo-acacia as a main active ingredient, which is obtained under optimal extraction conditions, as well as a method for preparing the same.

### BACKGROUND OF THE INVENTION

Generally, alcoholic drinks have appeared from the beginning of recorded human history and have been favored drinks until now. However, such alcoholic drinks can cause various problems, and among such problems, health problems caused by drinking alcohol are those most frequently pointed out. Namely, drinking alcohol is known to cause problems such as liver disease and reductions in stomach, colon and brain functions, and the appearance of such damage from alcohol have continued until the present time. Thus, research undertaken to protect the liver from alcohol and to relieve hangovers caused by drinking have been continuously conducted.

As used herein, the term “hangover” refers to various symptoms such as unpleasantness, headache and decrease in mental and physical ability to work, which occur when becoming sober and may last until the next day or even two days later after drinking alcohol. Such hangovers seem to occur because alcohol or acetaldehyde is accumulated in the human body due to lack of alcohol dehydrogenase or acetaldehyde dehydrogenase. In other words, when alcohol is ingested into the body via drinking, it is degraded by alcohol dehydrogenase while it is first converted into acetaldehyde and then into acetic acid, a metabolite thereof. If this metabolism does not occur smoothly, the accumulated ethyl alcohol or the acetaldehyde converted during the alcohol degradation process will lead to toxin processing, causing damages to liver and brain cells, so that metabolic disorders in the body will occur, causing whole body fatigue, abdominal distension, emesis, headache, and even chill and abdominal pain, resulting in hangovers.

Such hangovers make daily life abnormal and cause lack of energy, having severe effects not only upon the hangover person himself but also upon other persons. Thus, such hangovers incur significantly negative results in daily life.

From olden times, in folk remedies for relief of such hangovers, various foods have been used. Namely, foods, such as ox-blood soup, bean sprout soup, dried Pollack soup, shellfish soup, oysters, or juices such as that of Aegopodium podagraria L, radishes, cucumbers, leeks, spinach, lotus roots, arrowroots, pine needle juice, ginseng juice, and green tea leaves, have been believed to be effective in relieving hangovers. Particularly, green tea leaves have been thought to be greatly effective in relieving hangovers, since they contain polyphenol and thus can easily decompose acetaldehyde.

Furthermore, drink-type beverages for relieving such hangovers have been developed and sold. For example, Korean patent No. 0181168 discloses a natural tea for relieving hangovers and a method for preparing the same. The natural tea prepared according to this Korean patent contains an extract from, or a powder of the leaf, stem or root of Alnus japonica and Sorbus commixta as an active component, as well as given amounts of extracts from the fruit of Ligustrum japonicum Thunberg and the root of Pueraria. Also, Korean patent application laid-open No. 2001-019767 discloses natural tea for relieving hangovers and a method for preparing the same. The natural tea prepared according to this Korean patent publication contains Chinese medicinal materials, including Pueraria flowers, Pueraria roots, Liquordice roots, Atractylodes Rhizome White, dried orange peels, Alismatis Rhizoma, Lycium chinense, and ginger.

By such various methods, various drinks for relieving hangovers, each having a unique characteristic, have been developed.

However, in spite of such various efforts to relieve hangovers, there is still a need for the development of drinks or foods that can effectively relieve hangovers.

### SUMMARY OF THE INVENTION

Accordingly, an object of the present invention is to provide natural plants having excellent effects in relieving hangovers and revitalizing liver functions and to provide a natural tea having excellent effects in relieving hangovers and revitalizing liver functions by rapidly lowering blood alcohol concentration when taken before or after drinking alcohol, in which the natural tea contains an extract from these natural plants as a main active ingredient.

Another object of the present invention is to provide a natural tea, which has the effects of relieving hangovers and revitalizing liver functions and which allows the human body to be maintained at normal conditions before or after generation of a hangover, thereby providing health benefits.

Still another object of the present invention is to provide a method for preparing the natural tea according to the above objects.

To achieve the above objects, the present invention provides a natural tea, which has the effects of relieving hangovers and revitalizing liver functions and which comprises an extract from Robinia pseudo-acacia as a main active ingredient, and conventional additives.

It will be understood that the extract from Robinia pseudo-acacia, which is used in the present invention, includes all parts of Robinia pseudo-acacia, including the leaf, stem, flower, root and fruit of Robinia pseudo-acacia.

In the natural tea according to another embodiment of the present invention, the extract from the leaf, stem, flower and root of Robinia pseudo-acacia is contained as a main active component, to which at least one selected from Hedyotis diffusa, Amomi semen, Glycyrrhiza and Pueraiae Flos extracts can be added as a minor active component. However, the present invention is achieved by the main active component, and such minor active components are optional and not critical to the present invention.

In another embodiment, the present invention provides natural tea having the effects of relieving hangovers and revitalizing liver functions, which comprises a mixture of 10-80% by weight of an extract from Robinia pseudo-acacia and 20-90% by weight of Hedyotis diffusa.

In still another embodiment, the present invention provides a natural tea having the effects of relieving hangovers and revitalizing liver functions, which comprises a mixture of 10-65% by weight of an extract from Robinia pseudo-acacia, 20-80% by weight of Hedyotis diffusa, 10-60% by weight of Amomi semen, and 10-50% by weight of Glycyrrhiza.

Also, the present invention provides a method for preparing natural tea having the effects of relieving hangovers and revitalizing liver functions, the method comprising the steps of: washing the leaf, stem, flower and root of Robinia pseudo-acacia with water; extracting the water-washed material with alcohol as an extraction solvent at 50-150° C. for 1-10 hours to prepare a Robinia pseudo-acacia extract as a main active component; and adding conventional additives to the extract.

In the method according to the present invention, the extraction step is preferably conducted at a temperature of 50-150° C. If the extraction temperature is below 50° C., the active ingredients of Robinia pseudo-acacia will not be easily extracted. If the extraction temperature is above 150° C., an extraction effect will not be increased and the active ingredients can be modified. The extraction step is most preferably conducted at a temperature of 50-90° C.

In another embodiment of the present invention, the extract is centrifuged at 2,500-7,500 rpm to separate robinine.

In still another embodiment of the present invention, each component is dried in the shade and pulverized to a size of 50-100 meshes such that powder tea is provided.

Acacia, which is used as a main component in the present invention, is an evergreen belonging to the family Leguminosae of the order Rosales, dicotyledonous plants. About 500 species of acacia are found in the tropical and temperate zones of the world, particularly in Australia. Its leaves are double paripinnate compound leaves, each having very small leaflets and a peptiole that is flat or similar to a leaf. Also, its flowers have a yellow or white color, and an acacia often designated as Robinia pseudo-acacia is also native to North America.

Hedyotis diffusa, which is used in the present invention, is an annual herb belonging to the family Rubiaceae and grows to a height of around 30 cm. It is native to China, but is also found on Mt. Halla in island of Jejudo and on the swampy land of Baekun mountain in Jeolla-Namdo in Republic of Korea. It can be regarded as a recently discovered medicinal herb not described in olden Chinese medicinal books, such as Compendium of Materia Medica. It has been used in research and experiments in China since 40 years ago, when it was first described in the Guangxi Journal of Traditional Chinese Medicine just after the year 1945. Hedyotis diffusa has been widely known in the world, since its efficacy and action were described in detail in the book “Traditional Chinese Medicine Research” published by the Taiwan Center of Traditional Chinese Medicine.

Glycyrrhiza, which is used in the present invention, is a perennial plant belonging to the family Leguminosae of the order Rosales, which are dicotyledonous plants. Its root has a reddish brown color and is deeply penetrated into the ground. Its stem is angular and grows straight to a height of around 1 m. The stem has white fuzz closely distributed thereon and displays a light ash color. Also, the stem has pellucid dots spread thereupon. The leaf is an alternate, imparipinnate compound leaf with 1-17 leaflets, which are egg-like in outline and have a sharp end. Each of the leaflets is 2-5 cm long and 1-3 cm wide, has white fuzz and pellucid dots on both sides, and has no serrate. Examples of Glycyrrhiza includes G. glabra var. glandulifera, native to Siberia, and G. glabra, native to Spain. A similar species includes G. pallidiflora.

Pueraiae Flos, which is used in the present invention, refers to the Pueraria flower in Chinese medicine and has been mainly used as a drug for relieving alcoholic poisoning and for stopping the discharging of blood.

The inventive natural tea prepared as described above has the effects of relieving hangovers and revitalizing liver functions. This natural tea comprises either an extract from, or a powder of the leaf, stem, flower and root of Robinia pseudo-acacia distributed in the Orient, as a main active component, to which at least one selected from Hedyotis diffusa, Amomi semen, Glycyrrhiza, and Pueraiae Flos extracts, which act to neutralize poison, is added at a given amount. When taken before or after drinking alcohol, the inventive natural tea shows the excellent effects of relieving hangovers and revitalizing liver functions.

### BRIEF DESCRIPTION OF THE DRAWINGS

FIG. 1 is a graphic diagram showing a change in body weight of the experimental what was used during a test period;

FIG. 2 is a graphic diagram showing an effect of long-term alcohol administration on hematocrit content; and

FIG. 3 is a graphic diagram showing an effect of long-term alcohol administration on hemoglobin content.

### DETAILED DESCRIPTION OF THE INVENTION

The present invention will hereinafter be described in further detail by examples and test examples. It should however be borne in mind that the present invention is not limited to or by the examples.

### EXAMPLE 1

### Preparation of Acacia Extract

The leaf, stem, flower and root of Robinia pseudo-acacia were screened, and washed with water. A given amount of the water-washed material was extracted with alcohol at about 70° C. for 5 hours, to give acacia extract.

### EXAMPLE 2

The acacia extract prepared according to Example 1 was used as a main active component, to which conventional additives, such as a sweetening agent, spices and a coloring agent, were added, thereby preparing natural tea having the effects of relieving hangovers and revitalizing liver functions.

### EXAMPLE 3

Natural tea having the effects of relieving hangovers and revitalizing liver functions was prepared in the same manner as in Example 2, except that a mixture of 80% by weight of the acacia extract and 20% by weight of an extract from Hedyotis diffusa was used.

### EXAMPLE 4

Natural tea having the effects of relieving hangovers and revitalizing liver functions was prepared in the same manner as in Example 2, except that a mixture of 70% by weight of the acacia extract and 30% by weight of an extract from Hedyotis diffusa was used.

### EXAMPLE 5

Natural tea having the effects of relieving hangovers and revitalizing liver functions was prepared in the same manner as in Example 2, except that a mixture of 60% by weight of the acacia extract and 40% by weight of an extract from Hedyotis diffusa was used.

### EXAMPLE 6

Natural tea having the effects of relieving hangovers and revitalizing liver functions was prepared in the same manner as in Example 2, except that a mixture of 50% by weight of the acacia extract and 50% by weight of an extract from Hedyotis diffusa was used.

### EXAMPLE 7

Natural tea having the effects of relieving hangovers and revitalizing liver functions was prepared in the same manner as in Example 2, except that a mixture of 40% by weight of the acacia extract and 60% by weight of an extract from Hedyotis diffusa was used.

### EXAMPLE 8

Natural tea having the effects of relieving hangovers and revitalizing liver functions was prepared in the same manner as in Example 2, except that a mixture of 30% by weight of the acacia extract and 70% by weight of an extract from Hedyotis diffusa was used.

### EXAMPLE 9

Natural tea having the effects of relieving hangovers and revitalizing liver functions was prepared in the same manner as in Example 2, except that a mixture of 20% by weight of the acacia extract and 80% by weight of an extract from Hedyotis diffusa was used.

### EXAMPLE 10

Natural tea having the effects of relieving hangovers and revitalizing liver functions was prepared in the same manner as in Example 2, except that a mixture of 10% by weight of the acacia extract and 90% by weight of an extract from Hedyotis diffusa was used.

### EXAMPLES 11-36

Natural teas having the effects of relieving hangovers and revitalizing liver functions were prepared in the same manner as in Example 2, except that extracts from Robinia pseudo-acacia, Hedyotis diffuse, Amomi semen, Glycyrrhiza, and/or Pueraiae Flos were mixed with each other at a mixing ratio given in Table 1 below.

TABLE 1
(unit: wt %)

| | | Hedyotis | Amomi | | Pueraiae |
|---|---|---|---|---|---|
| | Acacia | diffusa | semen | Glycyrrhiza | Flos |
| | extract | extract | extract | extract | extract |
| Example 11 | 60 | 20 | 10 | 10 | 0 |
| Example 12 | 50 | 30 | 10 | 10 | 0 |
| Example 13 | 50 | 20 | 20 | 10 | 0 |
| Example 14 | 40 | 40 | 10 | 10 | 0 |
| Example 15 | 40 | 30 | 20 | 10 | 0 |
| Example 16 | 30 | 50 | 10 | 10 | 0 |
| Example 17 | 30 | 40 | 20 | 10 | 0 |
| Example 18 | 30 | 40 | 10 | 20 | 0 |
| Example 19 | 30 | 30 | 20 | 10 | 10 |
| Example 20 | 20 | 40 | 10 | 20 | 10 |
| Example 21 | 20 | 40 | 10 | 10 | 20 |
| Example 22 | 20 | 50 | 10 | 10 | 10 |
| Example 23 | 20 | 40 | 20 | 10 | 10 |
| Example 24 | 20 | 30 | 10 | 20 | 20 |
| Example 25 | 20 | 20 | 10 | 20 | 30 |
| Example 26 | 10 | 80 | 10 | 0 | 0 |
| Example 27 | 10 | 70 | 10 | 10 | 0 |
| Example 28 | 10 | 60 | 10 | 10 | 10 |
| Example 29 | 10 | 50 | 20 | 10 | 10 |
| Example 30 | 10 | 40 | 20 | 20 | 10 |
| Example 31 | 10 | 30 | 30 | 20 | 10 |
| Example 32 | 10 | 20 | 40 | 20 | 10 |
| Example 33 | 10 | 20 | 50 | 20 | 10 |
| Example 34 | 10 | 20 | 60 | 0 | 10 |
| Example 35 | 10 | 20 | 0 | 50 | 20 |
| Example 36 | 10 | 20 | 0 | 20 | 50 |

### TEST EXAMPLE 1

### Change in Body Weight

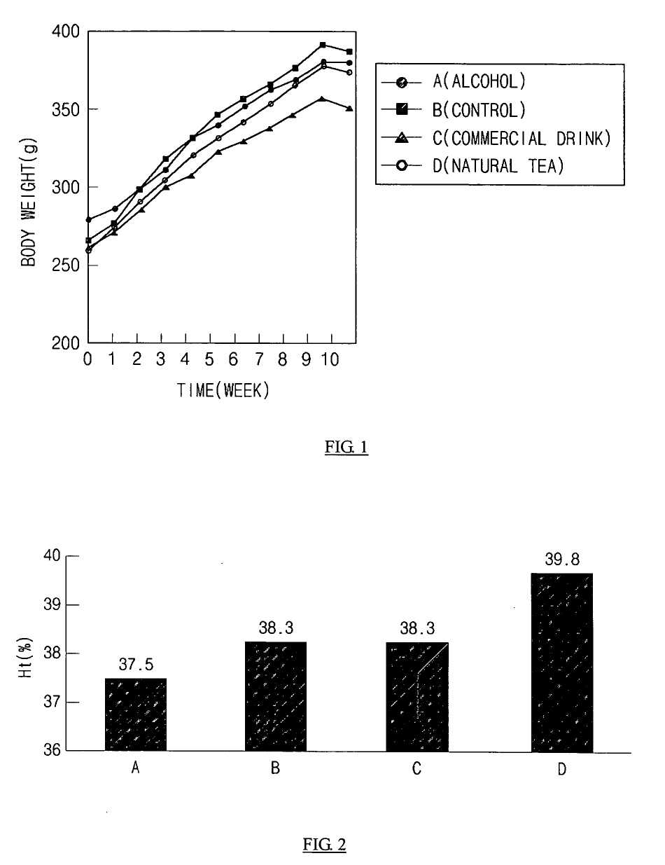

FIG. 1 shows a change in body weight during a test period. Up to seven weeks after test start, three test groups except for the control group B were administered only with ethanol in order to cause liver damage.

Upon test start, body weights were 278.9±13.7 g, 261.6±22.0 g, 260.6±23.6 g and 263.7±13.1 g for A, B, C and D groups, respectively. The body weight of the groups administered with alcohol was about 17 g higher than the control group.

The control group B, which was not administered with alcohol and drinks, showed the highest increase in body weight.

In spite of long-term ethanol administration, the alcohol-only group A showed a continuous increase in body weight during a test period as compared to the other two groups C and D administered with hangover relief drinks. Presumably, this is because the body weight of the group A upon the start of tests was higher than the other two groups C and D. The hangover relief drink-administered groups showed an increase in body weight up to three weeks after test start without a significant difference between the groups. However, after three weeks, group D, the group administered with the inventive natural tea, showed a clear increase in body weight as compared to the group C administered with usual hangover relief drinks in the market.

### EXAMPLE 2

### Hematocrit and Hemoglobin Contents

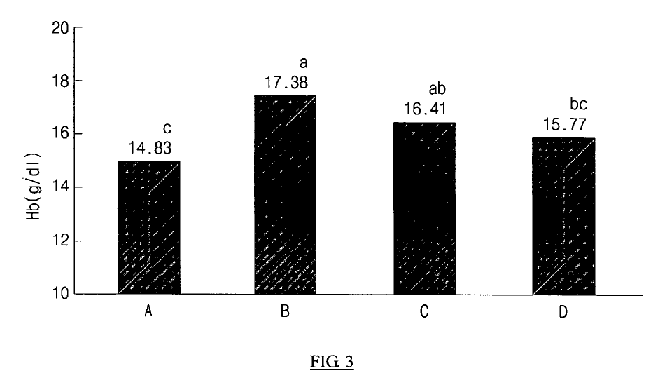

FIG. 2 and 3 show hematocrit (Ht) and hemoglobin (Hb) contents, respectively, which were measured upon the completion of the testing. There was no significant difference in Ht content between the treated groups. However, group D, the group administered with the inventive natural tea, showed the highest Ht content of 39.8%. The control group B and group C, the group administered with a usual hangover relief drink in the market, showed an Ht content of 38.0%, and group A, the group only administered with alcohol, showed a somewhat lower Ht content of 37.5%.

Hb contents were significantly different statistically between the treated groups (p<0.05). The control group showed the highest Hb content of 17.38 g/dl, the commercial hangover relief drink-administered group C showed 16.41 g/dl, the inventive natural tea-administered group D showed 15.77 g/dl, and the alcohol-only group A showed 14.83 g/dl.

### EXAMPLE 3

### Serum Lipid Content

Table 2 below shows serum lipid content measured upon the end of the test. The control group B showed the highest triglyceride (TG) content of 76.0 mg/dl. The commercial hangover relief drink-administered group C and the inventive natural tea-administered group D showed substantially similar TG contents of 53.6 mg/dl and 49.0 mg/dl, respectively.

Total cholesterol (TC) contents were in a range of 121.2-127.6 mg/dl without the difference between the treated groups. Regarding high-density lipoprotein cholesterol (HDL) contents, the inventive natural tea-administered group D showed the highest value of 39.6 mg/dl, and the commercial drink-administered group C showed the lowest value of 31.3 mg/ml (p<0.05).

Low-density lipoprotein cholesterol (LDL) contents were not different between the test groups, but the commercial drink product-administered group showed a somewhat higher value of 80.2 mg/dl. Atherogenic index (AI), which is a risk factor for adult diseases, was 2.14 in the inventive natural tea-administered group D, which is significantly lower statistically than the other treated groups (p<0.05).

TABLE 2
Effect of long-term alcohol administration on
serum lipid concentration (mean ± SD)

| Treated | Serum lipid (mg/dl) | | | | |
|---|---|---|---|---|---|
| groups | | | | | Atherogenic |
| (n = 9) | TG | TC | HDL | LDL | index |
| A | 58.5 ± 17.0 | 121.2 ± 7.9 | 33.3 ± 5.6 | 76.4 ± 8.9 | 2.75 ± 0.60 |
| B | 76.0 ± 30.0 | 127.6 ± 11.6 | 37.2 ± 6.9 | 75.2 ± 6.1 | 2.48 ± 0.37 |
| C | 53.6 ± 12.3 | 122.2 ± 7.6 | 31.3 ± 7.0 | 80.2 ± 9.4 | 3.10 ± 10.3 |
| D | 49.0 ± 16.7 | 123.5 ± 7.8 | 39.6 ± 4.0 | 74.1 ± 9.9 | 2.14 ± 0.32 |

* TG: triglyceride
TC: total cholesterol
HDL: high-density lipoprotein cholesterol
LDL: low-density lipoprotein cholesterol
Atherogenic index = (TC − HDL)/HDL

Superscripts indicated with different letters have shown significant difference between mean values (p<0.05).

### TEST EXAMPLE 4

### Liver Function Examination

Table 3 below shows the effect of long-term alcohol administration on liver functions. Bilirubin, ALP, GOT and r-GTP contents were statistically different between the treated groups (p<0.05). The bilirubin contents were 3.54 mg/dl, the highest value, in the control group B, and 1.21 mg/dl, the lowest value, in the inventive natural tea-administered group D. Alkaline phosphatase (ALP) activities were 17.7 K-A unit, the highest value, in the alcohol-only group A, and 11.9 K-A unit, the lowest value, in the natural tea-administered group D. Glutamic oxaloacetic transaminase (GOT) activities were 77.4 IU/L, the highest value, in the alcohol-only group A, and 60.7 IU/L, the lowest value, in the natural tea-administered group D. GPT activities were not significantly different between the treated groups but were somewhat lower in the inventive natural tea-administered group D. γ-glutamyl transpeptidase (γ-GTP) contents which are used to evaluate alcoholic fatty liver were 127.6 mU/ml, the highest value, in the commercial hangover relief drink-administered group C, and 122.6 mU/ml, the lowest value, in the alcohol-only group A, which is statistically lower than the commercial product-administered group C (p<0.05). Meanwhile, lactate dehydrogenase (LDH) activities were not significantly different between the treated groups. The control group B showed the highest LDH activity of 398.0 μ/L, and the inventive natural tea-administered group D showed the lowest LDH activity of 339.7 μ/L.

TABLE 3
Effect of long-term alcohol administration on liver functions

| Treated | | | | | | |
|---|---|---|---|---|---|---|
| groups | Bilirubin | ALP | GOT | GPT | r-GTP | |
| (n = 9) | (mg/dl) | (K-A unit) | (IU/L) | | (mU/ml) | LDH (μ/L) |
| A | 1.42 ± 1.02 | 17.7 ± 2.01 | 77.4 ± 16.6 | 37.4 ± 9.9 | 122.6 ± 54.7 | 375.3 ± 62.0 |
| B | 3.54 ± 2.01 | 13.8 ± 4.83 | 68.8 ± 7.3 | 34.6 ± 7.3 | 77.0 ± 34.4 | 398.0 ± 57.4 |
| C | 2.35 ± 1.05 | 15.4 ± 3.64 | 74.0 ± 9.8 | 37.3 ± 6.4 | 127.6 ± 48.5 | 353.9 ± 58.4 |
| D | 1.21 ± 0.63 | 11.9 ± 2.59 | 60.7 ± 10.1 | 31.8 ± 5.5 | 115.5 ± 41.7 | 339.7 ± 76.5 |

ALP: alkaline phosphatase (*K-A unit: King-Armstrong unit)
GOT: glutamic oxaloacetic transaminase
GPT: glutamic pyruvic transaminase
γ-GTP: r-glutamyl transpeptidase
LDH: lactate dehydrogenase

Superscripts indicated with different letters have shown significant difference between mean values (p<0.05).

### TEST EXAMPLE 5

### Blood Ethanol and Acetaldehyde Concentrations, and Alcohol Dehydrogenase (ADH) Activity

As given in Table 4 below, total alcohol intakes were zero in the control group B with no administration of alcohol and hangover relief drinks. Total alcohol intakes were 28.4 g in the alcohol-only group A, 26.4 g in the commercial hangover relief drink-administered group C, and 27.1 g in the inventive natural tea-administered group D. Thus, the total alcohol intakes were statistically different between the ethanol-treated groups (p<0.05).

Blood ethanol concentrations, which had been measured after long-term alcohol administration, were 0.015%, 0.010%, 0.004% and 0.003% as averages in the commercial hangover relief drink-administered group C, the alcohol-only group A, the control group B and the inventive natural tea-administered group D, respectively (p<0.05). Meanwhile, blood acetaldehyde concentrations were 82.6±36.2 μg/dl, 39.8±44.0 μg/dl, and 32.2±35.6 μg/dl in the commercial hangover relief drink-administered group, the inventive natural tea-administered group and the control group, respectively. Thus, the blood acetaldehyde concentration in the inventive natural tea-administered group was lower than the commercial hangover relief drink-administered group but similar to the control group. Also, the blood acetaldehyde concentration in the alcohol-only group A was 222.3±85.8 μg/dl which is much higher than the other three groups.

Alcohol dehydrogenase (ADH) activity was 17.98 nmol NADH/min/mg protein, the highest value, in the inventive natural tea-administered group D. ADH activities were 16.8 nmol NADH/min/mg protein in the commercial hangover relief drink-administered group C, 15.55 nmol NADH/min/mg protein in the control group B, and 15.00 nmol NADH/min/mg protein in the alcohol-only group A. Thus, the inventive natural tea-administered group D showed a somewhat higher ADH activity than the other three groups.

TABLE 4
Alcohol intake during test period, blood alcohol and acetaldehyde
concentrations, and ADH activity (mean ± SD)

| Test | | | | |
|---|---|---|---|---|
| group | Alcohol | Alcohol | Acetaldehyde | |
| (n = 9) | intake (g) | concentration (%) | (μg/dl) | ADH |
| A | 28.1 ± 1.21 | 0.010 ± 0.010 | 222.3 ± 85.8 | 15.00 ± 3.93 |
| B | 0 | 0.004 ± 0.003 | 32.2 ± 35.6 | 15.55 ± 3.41 |
| C | 26.4 ± 1.56 | 0.015 ± 0.007 | 82.6 ± 36.2 | 16.80 ± 2.40 |
| D | 27.1 ± 1.17 | 0.003 ± 0.003 | 39.8 ± 44 | 17.98 ± 2.97 |

(n = 9, mean ± SD)
* ADH: alcohol dehydrogenase (nmol NADH/min/mg protein)

Superscripts indicated with other letters have shown significant difference between mean values (p<0.05).

In Test Examples 1-5, alcohol was administered to white rats for a long period (10 weeks), and the alcohol degradation efficacies of the commercial product as a hangover relief drink and the inventive natural tea were examined. From the above reported results, it can be concluded the following;

1) Growth rates were somewhat higher in the control group but not statistically different between the treated groups. Hematocrit (Ht) values were higher in the natural tea-administered group than in the other groups but not statistically different between the treated groups. Hemoglobin contents were statistically different between the treated groups (p<0.05).

2) Triglyceride (TG) and HDL-cholesterol contents, and atherogenic indexes were significantly different between the treated groups (p<0.05).

TG contents were the lowest for the natural tea-administered group whereas HDL-cholesterol contents were the highest for the natural tea-administered group.

3) Liver function factors (bilirubin, ALP, GOT and γ-GTP) were significantly different between the treated groups (p<0.05).

Bilirubin contents were 3.54 mg/dl, the highest value, in the control group, and 1.21 mg/dl, the lowest value, in the inventive natural tea-administered group. ALP activities were 17.7 K-A unit, the highest value, in the alcohol-only group, and 11.9 K-A unit, the lowest value, in the inventive natural tea-administered group. GOT content were 77.4 IU/L, the highest value, in the alcohol-only group, and 60.7 IU/L, the lowest value, in the inventive natural tea-administered group. γ-GTP content, which are used to evaluate alcoholic fatty liver, were higher on the order of the commercial hangover relief drink-administered group, the alcohol-only group, the inventive natural tea-administered group and the control group.

4) Blood ethanol concentrations were 0.003%, the lowest value, for the natural tea-administered group (p<0.05), and blood acetaldehyde concentrations were 82.6±36.2 μg/dl and 39.8±44.0 μg/dl in the commercial hangover relief drink-administered group and the natural tea-administered group, respectively. Thus, blood acetaldehyde concentration was lower in the natural tea-administered group than in the commercial hangover relief drink-administered group. Meanwhile, alcohol dehydrogenase activities had no significance between the treated groups but were higher in the natural tea group than in the other groups.

To summarize, alcohol was administered orally to white rats for a long period (10 weeks) at an amount sufficient to cause alcoholic liver damage after which the inventive natural tea (Dawn-808), a hangover relief drink, was administered to the white rats from three weeks before the end of the test. In this case, the white rat group administered with the inventive natural tea showed excellent results for serum lipid concentration and liver functions as compared to the other groups. Furthermore, in the natural tea-administered group, ADH activity in liver tissue was somewhat higher than the other groups, and blood alcohol concentration was significantly lower than the other groups. This suggests that the inventive natural tea has the effect of promoting alcohol metabolism.

### TEST EXAMPLE 6

### Change in Blood Alcohol and Acetaldehyde Concentrations

Tables 5 and 6 below show blood alcohol concentrations measured in first and second clinical tests, respectively.

In the first clinical test, test volunteers following an occupation were drunk with at least bottle of Soju (distilled liquor). Table 5 shows blood alcohol concentrations together with the acetaldehyde concentrations in blood drawn at 12 hours (9:00 AM the next day) after drinking alcohol. The blood alcohol concentrations measured with an alcolmeter were not statistically different between a test group and a control group, but were lower in the inventive natural tea-drunk test group than in the test group with the passage of time. The blood alcohol concentrations measured at 12 hours after drinking alcohol were 0.006% and 0.030% in the test group and the control group, respectively. Meanwhile, the blood acetaldehyde concentrations measured at the same time as the blood alcohol concentrations in the test group was reduced to ½ lower level than that in the control group. Such results generally coincide with the test group volunteer's answer that the inventive natural tea helped to relieve hangovers on the next day after drinking alcohol.

TABLE 5
Effect of natural tea on blood alcohol and acetaldehyde concentrations

| | | | | | | Acetaldehyde |
|---|---|---|---|---|---|---|
| Volunteers | Blood alcohol concentration (%), | | | | | (%, ×10⁻⁴) |
| (n = 2) | 0 min | 60 min | 120 min | 150 min | 720 min | 720 min |
| Test group | 0.148 ± 0.049 | 0.117 ± 0.065 | 0.098 ± 0.051 | 0.058 ± 0.055 | 0.006 ± 0.007 | 7.72 ± 6.00 |
| Control group | 0.173 ± 0.065 | 0.169 ± 0.081 | 0.140 ± 0.066 | 0.082 ± 0.034 | 0.030 ± 0.032 | 15.78 ± 3.94 |

(first clinical test) (mean ± SD)

Table 6 shows the results of a second clinical test. In the second clinical test, university students drank one bottle of Passport®, a Korean whisky brand, and additional drinking of alcohol was prohibited. Blood alcohol concentrations were lower than those in the first clinical test where the test volunteers drank at least one bottle of soju, one kind of Korean alcohol beverage. Presumably, results of the second clinical test were as such because the amount of alcohol consumed was less and the age of the test volunteers was lower than the volunteers in the first clinical test.

Like the first clinical test, the test group administered with the inventive natural tea showed lower blood alcohol concentrations than the control group, except for a point 30 minutes after drinking alcohol. The blood alcohol concentrations at 360 minutes after drinking alcohol were 0.016% in the test group and 0.033% in the control group, indicating a great difference in blood alcohol concentrations between the two groups.

Meanwhile, blood acetaldehyde concentrations were measured 60 minutes, 150 minutes and 240 minutes after drinking alcohol, and the results are given in Table 7 below. The blood acetaldehyde concentration at 60 minutes after drinking alcohol had no significance between the two groups but was far lower in the test group than in the control group. This difference in blood acetaldehyde concentration was reduced at 150 minutes and 240 minutes after drinking alcohol. The acetaldehyde concentration had a correlation with the blood alcohol concentration.

TABLE 6
Effect of natural tea on blood alcohol concentration

| Volunteers | Blood alcohol concentration (%) | | | | | | |
|---|---|---|---|---|---|---|---|
| (n = 14) | 0 min | 30 min | 60 min | 120 min | 180 min | 270 min | 360 min |
| Test group | 0.107 ± 0.020 | 0.111 ± 0.049 | 0.087 ± 0.025 | 0.058 ± 0.037 | 0.042 ± 0.033 | 0.029 ± 0.031 | 0.016 ± 0.024 |
| Control group | 0.115 ± 0.013 | 0.107 ± 0.029 | 0.103 ± 0.013 | 0.101 ± 0.046 | 0.080 ± 0.042 | 0.057 ± 0.031 | 0.033 ± 0.026 |

(second clinical test) (mean ± SD)

TABLE 7
Effect of natural tea on blood acetaldehyde concentration

| | Acetaldehyde (%, ×10⁻⁴) | | |
|---|---|---|---|
| Volunteers (n = 14) | 60 min | 150 min | 240 min |
| Test group | 78.6 ± 33.67 | 60.35 ± 25.81 | 33.12 ± 12.70 |
| Control group | 104.62 ± 45.93 | 77.69 ± 39.08 | 43.03 ± 10.94 |

(second clinical test) (mean + SD)

### TEST EXAMPLE 7

### Change in Liver Function Activity

Table 8 shows a change in liver function activity of blood collected in the second clinical test. GOT and GPT are numerical values for enzymes which are transferred from liver cells to blood when the liver cells are broken due to inflammation in the liver. GOT which indicates the degree of breakdown of the liver cells was lower in the test group than in the control group with the passage of time. Particularly at 150 minutes after drinking alcohol, GOT was statistically significantly lower in the test group than in the control group (p<0.05).

γ-GTP, which is used to evaluate fatty liver and alcoholic liver diseases, was higher in the test group than in the control group at 0 minutes after drinking alcohol. However, γ-GTP in the test group was then reduced and reached a similar value to that in the control group at 240 minutes after drinking alcohol.

TABLE 8
Effect of natural tea on liver functions

| | | (mean ± SD) | | | |
|---|---|---|---|---|---|
| Measurement | | Liver function activity | | | |
| items | | 0 min | 60 min | 150 min | 240 min |
| GOT | Test | 63.4 ± 12.4 | 68.2 ± 12.6 | 41.8 ± 7.00** | 31.2 ± 7.3 |
| | group | | | | |
| | Control | 62.0 ± 7.5 | 89.4 ± 16.4 | 89.0 ± 9.4 | 40.1 ± 4.8 |
| | group | | | | |
| GPT | Test | 36.8 ± 5.9 | 49.6 ± 10.6 | 41.4 ± 12.4 | 24.1 ± 7.5 |
| | group | | | | |
| | Control | 45.6 ± 6.0 | 57.3 ± 11.6 | 56.7 ± 9.4 | 33.1 ± 4.5 |
| | group | | | | |
| γ- | Test | 45.9 ± 10.2 | 35.9 ± 3.1 | 32.4 ± 3.2 | 27.4 ± 8.5 |
| GTP | group | | | | |
| | Control | 34.7 ± 3.0 | 60.6 ± 16.6 | 58.4 ± 12.7 | 28.6 ± 1.9 |
| | group | | | | |

**(p < 0.05)
GOT & GPT (Kamen unit)
γ-GTP (mU/ml)

### TEST EXAMPLE 8

### Change in Serum Lipid

Table 9 below shows serum triglyceride (TG) and total cholesterol (TC) contents.

Blood TG content was lower in the test group than in the control group at all the time point of measurements. This seems attributable to a difference in TG content between the two groups upon the start of measurement. However, the serum TG content in the control group reached the highest value of 354.3 mg/dl at 150 minutes after drinking alcohol and then was reduced by about 100 mg/dl at 240 minutes after drinking. In both the two groups, TG contents were higher at 240 minutes than at 0 minute. Blood TC contents were reduced in both the two groups with the passage of time after drinking alcohol.

TABLE 9
Effect of natural tea on serum lipid content

(mean ± SD)

| Measurement | | Serum lipid (mg/dl) | | | |
|---|---|---|---|---|---|
| items | | 0 min | 60 min | 150 min | 240 min |
| TG | Test | 204.4 ± 42.8 | 229.0 ± 34.0 | 226.2 ± 32.5 | 224.8 ± 57.0 |
| | group | | | | |
| | Control | 233.8 ± 49.1 | 261.8 ± 55.3 | 354.3 ± 56.2 | 255.2 ± 33.7 |
| | group | | | | |
| TC | Test | 162.5 ± 6.7 | 166.3 ± 8.8 | 160.1 ± 9.8 | 142.2 ± 10.0 |
| | group | | | | |
| | Control | 188.5 ± 12.6 | 177.2 ± 13.0 | 183.8 ± 14.3 | 134.5 ± 8.3 |
| | group | | | | |

TG: triglyceride
TC: total cholesterol

### TEST EXAMPLE 9

### Change in Serum ADH Activity

Alcohol dehydrogenase activity having a determinative effect on alcohol degradation follows zero order reaction where alcohol is metabolized only at a constant amount per unit time regardless of the alcohol concentration in cells. Thus, the activity of this enzyme is constant regardless of the degree of drinking (Takagi et al., 1985; and Oshii et al., 1973).

Table 10 below shows a change in ADH activity with time.

The ADH activities measured at 0 min. after drinking alcohol were 2.63 U/L and 3.53 U/L in the test group and the control group, respectively, indicating that the ADH activity in the control group was higher than the test group but had no significant statistical difference from the test group. However, regarding a change in ADH activity with time, the test group showed an ADH activity of 4.12 U/L at 60 minute after drinking alcohol, which indicates more than 50% increase in ADH activity as compared to 2.63 U/L at 0 minute. Also, the test group showed an ADH activity of 3.76 U/L at 150 minutes and an ADH activity of 3.09 U/L at 240 minutes, which is about 17% higher than the ADH activity at 0 minute after drinking alcohol. On the other hand, in the control group, the measured ADH activity values at 150 minutes and 240 minutes were somewhat inconstant, but the ADH activity at 0 minutes after drinking was reduced with the passage of time and reduced by more than 20% at 240 minutes after drinking alcohol.

TABLE 10
Effect of natural tea on ADH activity

| | (mean ± SD) | | | |
|---|---|---|---|---|
| Groups | ADH activity (U/L) | | | |
| (n = 14) | 0 min | 60 min | 150 min | 240 min |
| Test group | 2.63 ± 0.42 | 4.12 ± 1.43 | 3.76 ± 0.83 | 3.09 ± 0.89 |
| Control group | 3.53 ± 0.83 | 3.49 ± 0.77 | 2.67 ± 0.63 | 2.76 ± 0.64 |

As can be seen in Table 10, although there was no statistical difference between the two groups, the test group which ingested the inventive natural tea showed a somewhat higher ADH activity than the control group. This suggests that the inventive natural tea promoted blood alcohol metabolism.

In Test Examples 6-9 above, the test volunteers were administered with the inventive natural tea before or after drinking alcohol, and measured for their alcohol degradation ability in a crossover experimental design. The test results provide the following possible conclusions.

1) Blood alcohol concentrations at all the time points of measurement were lower in the inventive natural tea-administered test group than in the control group but had no statistical significance between the two groups. Also, blood acetaldehyde concentrations were lower in test group than in the control group.

2) Regarding liver function activity, GOT concentration became lower in the test group than in the control group with the passage of time. Particularly, at 150 minutes after drinking, there came to be a statistical significance between the two groups (p<0.05).

GPT and γ-GTP concentrations reached the highest values at 60 minutes after drinking and then declined. GPT and γ-GTP concentrations were somewhat lower in the natural tea-administered test group than in the control group.

3) Regarding serum lipid content, triglyceride (TG) content was higher in the control group than in the test group. Particularly in the control group, TG content reached the highest value at 150 minutes and then declined by about 100 mg/dl at 240 minutes. In both the two groups, the TG content at 240 minutes was higher than at 0 minutes.

Total cholesterol (TC) contents were reduced in both the two groups with the passage of time after drinking alcohol.

4) Meanwhile, in a question survey conducted on the volunteers who participated in the clinical tests, the respondents answered that hangover syndromes, such as headache, heartburn and asthenia, which are usually experienced on the next day after drinking alcohol, were relieved after taking the inventive natural tea.

As described above, the present invention provides the natural powder or liquid tea comprising an extract from Robinia pseudo-acacia, and optionally, a given amount of extracts from Hedyotis diffusa, Amomi semen, Glycyrrhiza and/or Pueraiae Flos, as well as the method for preparing the same. The natural tea according to the present invention shows the effects of relieving hangovers and protecting the liver when taken before or after drinking alcohol. Particularly, according to the present invention, robinine extracted from Robinia pseudo-acacia can be used as a single active ingredient in a hangover relief drink.

1. Natural tea having the effects of relieving hangovers and revitalizing liver functions, the natural tea comprising an extract from Robinia pseudo-acacia as a main active ingredient and conventional additives.

2. The natural tea of claim 1, which additionally comprises at least one selected from Hedyotis diffusa, Amomi semen, Glycyrrhiza and Pueraiae Flos extracts, as a minor active ingredient.

3. The natural tea of claim 1, which comprises a mixture of 10-80% by weight of the Robinia pseudo-acacia extract and 20-90% by weight of the Hedyotis diffusa extract.

4. The natural tea of claim 2, which comprises a mixture of (i) 10-65% by weight of the Robinia pseudo-acacia extract, (ii) 20-80% by weight of the Hedyotis diffusa extract, and (iii) 10-60% by weight of the Amomi semen extract, or 10-50% by weight of the Glycyrrhiza extract.

5. The natural tea of claim 2, which is prepared by extracting 10-30% by weight of the Robinia pseudo-acacia, 20-60% by weight of the Hedyotis diffusa, 10-40% by weight of the Amomi semen, 10-20% by weight of the Glycyrrhiza and 10-30% by weight of the Pueraiae Flos, with an extraction solvent.

6. Natural powder tea having the effects of relieving hangovers and revi-talizing liver functions, the natural powder tea being prepared by washing the leaf, stem, flower and root of Robinia pseudo-acacia with water, drying the water-washed material in the shade, and pulverizing the dried material to a size of 50-100 meshes.

7. The natural powder tea of claim 6, which additionally comprises a 50-100 mesh size powder of at least one selected from Hedyotis diffusa, Amomi semen, Glycyrrhiza and Pueraiae Flos.

8. The natural powder tea of claim 7, which is prepared by pulverizing (i) 10-65% by weight of the Robinia pseudo-acacia, (ii) 20-80% by weight of the Hedyotis diffusa, and (iii) 10-60% by weight of the Amomi semen, or 10-50% by weight of the Glycyrrhiza, to a uniform size of 50-100 meshes.

9. The natural powder tea of claim 7, which is prepared by pulverizing 10-30% by weight of the Robinia pseudo-acacia, 20-60% by weight of the Hedyotis diffusa, 10-40% by weight of the Amomi semen, 10-20% by weight of the Glycyrrhiza and 10-30% by weight of the Pueraiae Flos, to a uniform size of 50-100 meshes.

10. A method for preparing natural tea having the effects of relieving hang-overs and revitalizing liver functions, the method comprising the steps of: washing the leaf, stem, flower and root of Robinia pseudo-acacia with water; extracting the waster-washed material with an alcohol extraction solvent at 50-150° C. for 1-10 hours to give an extract to be used as an active ingredient; and adding conventional additives to the extract.

11. The method of claim 10, which additionally comprises adding at least one minor active ingredient selected from Hedyotis diffusa, Amomi semen, Glycyrrhiza and Pueraiae Flos extracts, to the Robinia pseudo-acacia extract.

12. The method of claim 10, wherein 10-80% by weight of the Robinia pseudo-acacia extract and 20-90% by weight of the Hedyotis diffusa are mixed with each other.

13. The method of claim 11, wherein (i) 10-65% by weight of the Robinia pseudo-acacia extract, (ii) 20-80% by weight of the Hedyotis diffusa extract, and (iii) 10-60% by weight of the Amomi semen extract or 10-50% by weight of the Glycyrrhiza extract are mixed with other.

14. The method of claim 11, wherein 10-30% by weight of the Robinia pseudo-acacia, 20-60% by weight of the Hedyotis diffusa, 10-40% by weight of the Amomi semen, 10-20% by weight of the Glycyrrhiza and 10-30% by weight of the Pueraiae Flos are mixed and then extracted with the extraction solvent.

15. The method of claim 10, which additionally comprises centrifuging the Robinia pseudo-acacia extract at 2,500-7,500 rpm to separate robinine for use.

16. The method of claim 10, which additionally comprises centrifuging the extracts at 5,000-7,500 rpm to separate active ingredients for use.
